(19) United States
(12) Patent Application Publication
(10) Pub. No.: US 2007/0276202 A1
(43) Pub. Date: Nov. 29, 2007

(54) Method and Apparatus for Measuring and Monitoring Vital Functions or Presence

(76) Inventors: Heikki Raisanen, Jyvaskyla (FI); Tomi Virtanen, Jarvenpaa (FI)

Correspondence Address: KATTEN MUCHIN ROSENMAN LLP, 575 MADISON AVENUE, NEW YORK, NY, 10022-2585, US

(21) Appl. No.: 11/631,272

(22) PCT Filed: Jul. 1, 2005
(86) PCT No.: PCT/FI05/00309

§ 371 Date: Mar. 21, 2007

(30) Foreign Application Priority Data
Jul. 1, 2004 (FI) 20040914

(51) Int. Cl. A61B 5/00; A61B 5/08

(52) U.S. Cl. 600/301; 600/527; 600/534

(57) ABSTRACT

A procedure for the monitoring of a person or an animal on a base (2) with a device that has at least one plane like sensor element (3) arranged to it for transforming the mechanical forces caused by the measured movements and/or the vital signs to electric signals and a signal handling device (7) that registers the movements and/or the respiration frequency and/or the pulse frequency and/or the presence on a base with the help of the signals obtained from the sensor element, characterised in that that after the beginning filtering the sensor signal is divided to n subchannels, an absolute value is taken from the signal in the subchannels, every detected signal is low pass filtered so that highest measurable pulse and/or respiration frequency can be determined, every detected signal is high pass filtered so that lowest measurable pulse and/or respiration frequency can be determined, from these detected signals the intervals between the crossings of the zero points are measured, the alteration of the intervals is measured, from the sub channels the one with the least alteration is chosen, this interval is used to point out the pulse and/or respiration frequency.

This invention relates to a method for monitoring a person's vital functions and presence lying on a foundation and an alarm and monitoring apparatus having one or several sensor elements arranged on the foundation in order to transform the mechanical forces caused by the person's movements and vital functions to electrical signals, and a signal processing and alarm device.

In the U.S. Pat. No. 5,902,255 a device is presented, which is capable, to monitor the body movements of a person placed on a chair or a bed with a piezoelectric element. The device determines the person's body movements or his presence on a chair or a bed by detecting body's fine movements in heartbeats or respiration. If the person is placed on bed, the counting of time is started with a timer, after which the measurement of time is reset by presuming the absence and the body movements. The presumption is that the person has fallen in sleep when the measured time passes the preset time. In U.S. Pat. No. 5,448,996 a level sensor device that monitors the patient's state of condition such as respiration, heartbeat and body movements is presented. The level sensor device constitutes of sensor element plates in the shape of rectangle that have horizontal piezo electrical sensor strines. The sensor element plates are placed in a patient's bed. Single Sensor mats can also be in the shape of circle or square for example. In the FI-patent 92139 a device that is placed around the patient 's wrist is presented. This device attempts to measure the alterations in person's condition of health by supervising for example the movement activity of the person. Also reference is made to U.S. pat publication 20040111045.

The so-called electret field, in another words the permanent electric charge injected to the dielectric material by ionisation is based on the locking of ions to the molecules and crystal structure. In the article: “EMF-polymer transducer as a detector of respiration in humans” by J. Siivola, K. Leinonen and L. Räisänen (Medical and Biological Engineering & Computing, November 1993), a certain shield that is adequate for the patient sensors active electromechanical material is introduced. The shield is based on electric field. The used shield such as polypropylen is a dielectric plastic shield, that constitutes of flat or tom up gas blisters that creates an electric shield (so called electret bubble film). Unlike the piezoelectric PVDF-shield, which is sensitive to bending, the electret bubble film made of gas bubbles is extremely sensitive to the alterations of thickness. This is an advantage for the adaptation of the invention.

There are a numerous amount of different devices on the market that monitor the presence of a person in bed and are capable to alarm a nurse or other responsible when the patient leaves the bed. Usually these devices are based on obtaining switching contact from a sensor that is placed on the bed under the sheets. The market have been lacking a device that can simultaneously collect measured data from a person's average pulse, respiration frequency and movement activity, in a wanted time period (for example 1 or 3 minutes), to save the collected data for the purpose of later and longsighted surveillance and to alarm if the changes in given limit values are exceeded or the person leaves the base. There are a lot of difficulties in developing the device to function in different circumstances. The greatest difficulty is the calculation algorithms that can adapt to different size persons and different kind of beds. When the circumstances change (a mattress or a person), also the handling of algorithms with which the pulse, respiration and movement activity is measured, has to be able to change.

The purpose of this invention is to create a more developed procedure device to confirm the presence of a person on a base, to measure vital signs with a dynamic sensor attached to a base and to monitor the changes in the condition of health according to the measured results. The object of the invention is specially to solve the questions around the signal handling which insures a flawless, desired functioning in different circumstances.

The invented device in capable to register vital functions, to show and report the changes in them on a wanted time period and to alarm if the given limit measurements differentiate with the actual measurements. The device is also Registering a persons presence/absence on a base and creates reports and alarms considering it. The invented device can be used for example in hospitals, elderly care or prisons but also at the homes of an elder person who is living alone. Typically the device is made so that it can also function as a nurse-calling device for the person who is monitored. It can also have a two directional talking connection line between the caretaker and the person taking cared of.

With the invented method any information about the persons condition of health, the intensity of his dream and the durance of the deep sleep correlated by the body movements and other patient's health related things can be monitored without any sensors or measurement devices needed to attach to the patient. With the procedure one can also be notified of the presence or absence of person on a base.

The invented device and procedure is suitable for long term monitoring when collecting information about some medicines long term effects are wanted for example. The invented signals handling is suitable also to be used in arrest cells and prisons where the problem often is that the arrested persons are usually under the influence of alcohol or drugs. In these circumstances especially their respiration should be monitored without being forced to attach any sensors or measuring devices to them. The invented procedure and sensor device is presented more detail in the enclosed claims.

The characteristic features of the method and sensor apparatus according to the present invention are in detail presented in the enclosed claims.

In the following the invention is presented more detail with the help of an example and a drawing in which:

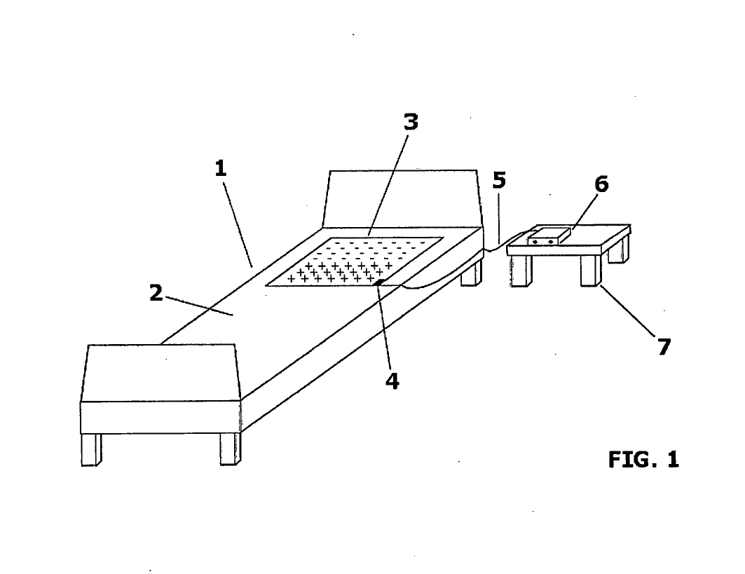

FIG. 1 presents the bed with the invented sensor device,

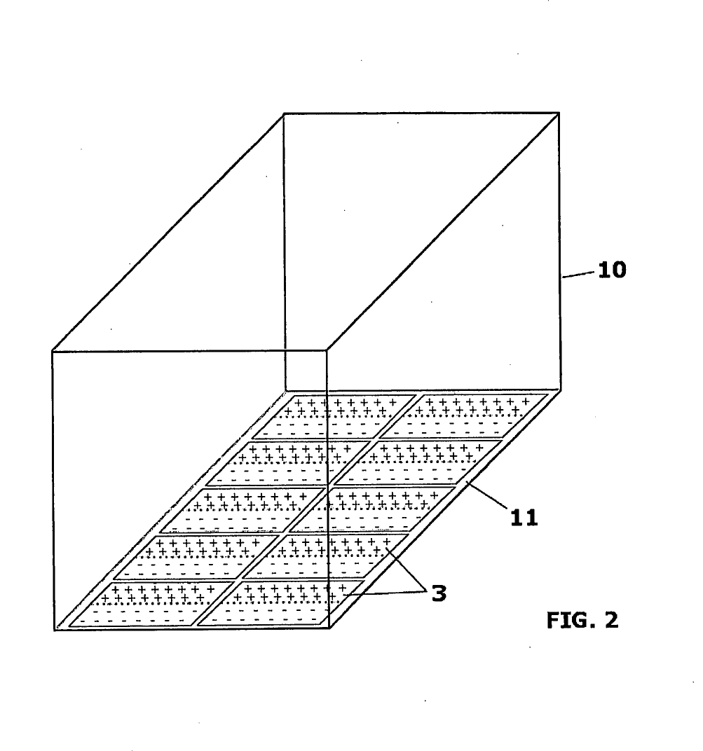

FIG. 2 presents a room to where an invented sensor device is placed on the floor.

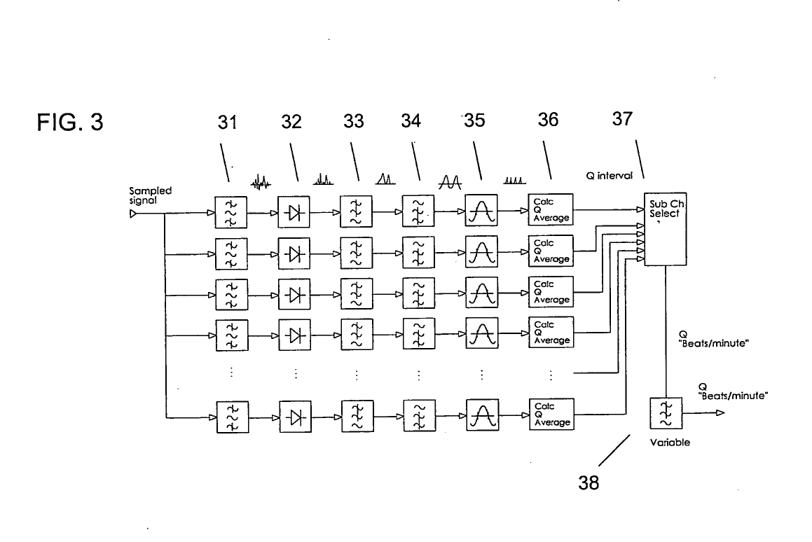

FIG. 3 presents the invented signal-handling device that measures an accurate respiration and pulse frequency from the signal coming from the dynamic sensor placed on the base.

FIG. 1 presents a bed 1 that has a mattress 2 as the patient's base. Under the bed a dynamic sensor element 3 has been placed that produces an electric signal. Instead of a bed a room floor can for example operate as the base in which case several sensors has been placed under the surface layer of the floor. The heartbeat, the frequency of the respiration and the body movements can be measured with the following manner from the electric signal. The mechanical forces caused by the vital signs and movements are turned into corresponding electric signals with a sensor. The electric signals are transformed to the electric signal-handling device 7 on the table 6 with the joint 4 and the connecting cable 5 connected to it on the side of the sensor 3. The connecting cable 5 can also be replaced with a wireless transmitter integrated to the sensor.

Planar sensor element 3 is for example a sensor made of electret bubble film in the shape of rectangle that reaches almost from side to side of the mattress 2. The patient is lying down so that the sensor element 3 is placed under the upper body, specially the lung. If the whole floor space of the room 10 is want to be used as the measuring ground (FIG. 2) then several room broad sensors 3 are placed under the surface of the floor 11. The accuracy of the device can be improved by using a signal-handling device with 12 channels in which there is a separate entrance to every sensor. The accuracy of the device can be improved by constructing it so that it has multiplexing between all the channels and an automatic locking system to what ever channel the strongest signal level occurs in the wanted frequency area.

The electric control unit 7 measures constantly the movements and vital signs (pulse, respiration) of the person upon the sensor while being on the base and registering the average pulse and respiration frequency at the chosen time periods with the help of follow-up algorithms. The electric control unit 7 consists of minimum one analogous preamplifier channel, A/D converter, micro controller and an information's processing, saving and/or broadcasting unit. It's also possible to measure moving activity from which is possible to determine is the dream peaceful or restless for example. From the combined measurement of the later can also be seen the time spent on the base and the time of leaving it. With the algorithms the cumulative sum of electric signals caused by person's movements that go over the preset level can be measured in the wanted time period, for example in 5 minutes periods. When there is a lot of movement, the sum is big. If the dream is restless and no falling in to REM dream occurs, the moving is dense. If the dream is peaceful, the very small electric signals caused by the respiration and pulse keep the moving activity small. There can also be set a level that the electric signals must go over should they register to the cumulative summa. The movements separate from the respiration and pulse can be monitored from the same sensor by using a multi channel preamplifier. The wanted signals can be brought up distinctly by using appropriate filters in each preamplifier channel or microcontroller part.

With the invented device constant information can be registered from the respiration and pulse frequency whenever the movements of the person won't make the measurement more complex. The device saves the measurement figures to the memory. If the device detects a greater contrasting figure comparing to the preset average figures, can an alarm be given to the care taking crew with the micro controller program. In addition conclusions about the patient's condition of health can be made from the period with a risen moving activity figure and from the average pulse level of the following period. The information is helpful when the differences in the patient's condition of health are observed in the long term even though the time periods average results wouldn't be used in the benefit of alarming.

A person's moving activity can be determined for example in the following manner. The preamplifier signal connected to the sensor 3 is measured with an appropriate sampling frequency with the help of A/D-converter. The amplitude of the signal alters on the both sides of the basic level comparable to the changes of power affecting to the sensor. The measurement time has been divided into measurement periods whose length is T. To each of the time periods an activity calculator C(k) is connected, where k=1, . . . N. In the beginning of the measuring period the activity calculators are set to zero. In case the total value of the amplitude exceeds the preset threshold level, the activity calculator C(k) related to the period in question is raised with one. The algorithm is there for measuring the sensor affecting changes of forces (movements) that have gone over the threshold level under a certain time period. The maximum figure for the activity calculator is T [s]×f [Hz], where the f is the sampling frequency.

It is also possible to use several thresholds levels in which case all the signal amplitudes that have gone over the threshold level are registered to separate activity calculators. This means that the movement activity can be grouped according to the power of movements. The measured figures of movement activity calculators can also be raised with the measured signal amplitudes total value instead of 1. In this case the figures of the calculators are depending quite strongly on the power of the movement instead of the amount of the movements.

A humidity and/or temperature measuring sensor or sensors can also be combined to the sensor 3. This makes it possible for the device 7 to gather information to the memory from the changes in person's temperature and the humidity of the bed.

The presented movement activity calculator algorithm can also be used for example to alarm if the patient lying down in bed is threatened by a bed sour as a result from over length rest. In this case a follow-up algorithms that resets every time when the movements go over a certain level can be set. This allows determining if the patient's movement has been sufficient and there is no risk of bed sour. After the reset the device start the measuring period from the beginning. If there isn't enough of moving for example in two hours, the device alarms a nurse.

The problem with detecting a persons presence on a base with a dynamic sensor has been to get accurate information about when the person is on the base, for example on a bed and in return, when he abandons it. This has a great importance when the device is been used in an elder care or a hospital where the attempt is to notify the nurses when a patient wakes up and gets on a move. The dynamic sensor attempts to react also to the environment signals such as the alterations of air pressure because the dynamic sensor produces an electric signal out of very small pressure changes. When monitoring a person's presence/absence on a base, we have achieved the best results with invented device by integrating a total valued (rectified) signal that is compared to some level. In another words by examining what is the signal's overall strength on a certain frequency level, typically 1 . . . 50 Hz. 1.

Particularly problematic is the need to fine-tune the filters and the algorithms on a different way for the different kind of beds when measuring a person's pulse or respiration frequency. As a solution to the problem multiple filters and algorithms has been placed to the device from which the appropriate one is either manually chosen or the device itself searches the most convenient one and locks itself to it. The algorithm can have a characteristic to independently monitor the measured result and is capable to detect if it's not accurate. In this case it can start a new search for more appropriate filter.

In the invented procedure for example to measure the pulse frequency after the starting filtering the signal is divided into n pieces sub channels. All the sub channels are filtered with different band pass filter 31 (FIG. 3). The band pass filters are chosen so that their pass bands are crossing each other a little bit.

The band pass filtered canals are detected so that total value 32 is taken from the signal and by the low pass filtrating 33 this signal. This procedure is corresponding to the full wave rectifying of the analogy electronics. In this the highest measurable pulse frequency is defined. The signal is high pass filtered 34 and is given to the detector 35 of point zero overdraft. In this the lowest measurable pulse frequency. The touching moments of the detector of the zero point are saved to a bumper that keeps inside n pieces numbers from which the pulse frequency 36 can be measured. The intervals 37 between the overdrafts of the zero points are measured from the signal. If the environment is not very disturbing the total time of two intervals is the same than the pulse interval. The amount of disturbances can be estimated from the alteration of the measured pulse interval. The more disturbance the more alterations in the pulse interval most likely. The result of the sub channel with the lowest alteration in the pulse interval is used. The measurement result of the pulse interval is then finally led to the low pass filtration 38. With this procedure the possible inconsistencies of the measurement results in the switch of sub channels can be balanced. In the aforementioned low pass filtration a changing border frequency can also be used. The border frequency is tuned according to how altering the measuring results of the pulse interval are. The border frequency is measured if the measuring results are changing a lot. With this a more stable and final result for the pulse frequency is received also in bad and disturbing conditions. The mentioned disturbances are usually distortion of signal between the pulse happening (heart) and the sensor including the body of the monitored person and other intermediate agents such as the mattress, chairs cushion etc. band pass filters are needed because specially the body movement caused by the heartbeat is transformed differently to the sensor depending of the person, the position or other intermediate agents (mattresses, cushions etc) and it is discovered that by monitoring relatively narrow frequency spectrum, a reasonably reliable pulse frequency is obtained. A corresponding procedure can also be applied to measure the respiration frequency.

The invented device can be equipped with a sufficient memory and for example with the help of the serial port in it, the registered information can be printed out. By continually averaging the respiration and or the pulse frequency of the person for example in the bed and registering the results from long time period, for example two weeks, the changes can be monitored. There can also be made assumptions whether the person's condition of health is going to change for better of worse. It is possible to integrate a GSM modem to the device. With the help of the GSM modem the collected information can be saved on an external server and the information can be handled with a separate browser-based program trough the Internet. In this procedure the GSM modem is used by sms based data transfer and the so-called SMS-gateway service or by transferring data continually with the so-called GPRS connection. In the hospital and care treatment the device can also functions as a calling device. With the help of the GSM modem a talking connection between the person in the bed and the nurse can be arranged. Instead of the GSM modem the wireless talking connection can also be arranged by for example integrating a so-called VOIP modem to the device. With the VOIP modem also the registered information can be transferred. The person can independently call for the nurse or open a talking connection with a separate push button, also a wireless one, included to the device if wanted. The visit of a nurse can be registered to the device with an integrated RFID device. In this case it can be arranged so that only the nurse can acknowledge the set on alarm or to make changes to the settings of the device. An oxygen oxide metering unit can also be included to the device so that the respiration and blood oxygen can be monitored simultaneously for example when doing a sleep apnea examination.

There can be a special advantage of monitoring the development of the averaged respiration and pulse frequency on a long term if there is a need to monitor the development of the rest pulse or the average respiration frequency of numerous persons wirelessly or automatically on the course of a long time period. This could be the case for example when testing the influence of a heart medicine for a larger group of patients at home.

Different details of the invention can also be used in the measuring of animals. When pigs are examined for example, the sensors and the device can be used on a corresponding manner when placed under the floor surface. This is useful when the moment of the labour of the pigs is wanted to be detected as well as in different kind of animal testing.

It is obvious to a person skilled in the art that the different kind of applying methods of this invention do not limit them self only to the forms presented but they alter according to the patent claims presented later on. Instead of the micro controller digital signal processors or controllers or other corresponding devices can be used. The preamplifier, a/d converter and processor can be separate components or integrated to the same circuit.

1. A procedure for the monitoring of a person or an animal on a base (2) with a device that has at least one plane like sensor element (3) arranged to it for transforming the mechanical forces caused by the measured movements and/or the vital signs to electric signals and a signal handling device (7) that registers the movements and/or the respiration frequency and/or the pulse frequency and/or the presence on a base with the help of the signals obtained from the sensor element, characterised in that:
1.) After the beginning filtering the sensor signal is divided to n subchannels.
2.) An absolute value is taken from the signal in the subchannels,
3.) Every detected signal is low pass filtered so that highest measurable pulse and/or respiration frequency can be determined,
4.) Every detected signal is high pass filtered so that lowest measurable pulse and/or respiration frequency can be determined,
5.) From these detected signals the intervals between the crossings of the zero points are measured.
6.) The alteration of the intervals is measured,
7.) From the sub channels the one with the least alteration is chosen.
8.) This interval is used to point out the pulse and/or respiration frequency.

2. The procedure according to the claim 1, characterised in that the pass bands of the pass band filters are slightly overlapping.

3. The procedure according to the claim 1, characterised in that it includes 4 or more subchannels for different kind of bases or persons being measured.

4. The procedure according to the claim 1, characterised in that the device chooses automatically the sub channel that fits the best on the moment.

5. The procedure according the claim 1, characterised in that the most convenient sub channel is chosen manually by the user.

6. The procedure according to the claim 1 defines the average movement activity and/or respiration frequency and/or pulse frequency produced by the electric signals caused by the movements and vital signs of the measured person.

7. The procedure according to the claim six has a signal handling part in which the measuring time is divided into measurement periods (T). In the measurement one or more activity calculators (C(k)) are used. In the beginning of every measurement the calculator is reset to zero and in case the amplitude of the total value of the signal exceeds the preset threshold level, the activity calculator associated to the measurement period is raised.

8. The procedure according to the claim 7 has an signal handling part in which several threshold levels are defined and the signal amplitudes that has exceeded each separate threshold levels are registered to their own activity calculators.

9. The procedure according to the claim 1, characterised in that time periods with a risen movement activity grade are determined in it's signal handling part by the electric signals caused by the movements of the measured subject. After this the calculation is reset to zero by itself and it begins a new time period to measure the movement activity.

10. A device to monitor a person or an animal on a base (2) with at least one customary sensor element (3) placed to the base to transfer the mechanical forces caused by the movement and/or the vital signs of the measured person into electric signals and a signal-processing device (7) that registers the movements and/or the respiration frequency and/or the pulse frequency and/or the presence on a base with the help of signals obtained from the sensor element, the method is characterised in that:
After the beginning filtering the sensor signal is divided into n pieces sub channels.
In the sub channel a total value is taken from the signal.
Every detected signal is low pass filtered so that the highest measurable pulse or respiration frequency can be determined.
Every detected signal high pass filtered so that the lowest measurable pulse or respiration frequency can be determined.
From these detected signals the intervals between the crossings of the zero points are measured.
The alteration of the intervals is measured.
From the sub channels the one with the least alteration is chosen.
This interval is used to point out the pulse and/or respiration frequency.
The sensor is dynamic.

11. The device according to the patent claim 10 has threshold value positioning organs that are used to define at least one threshold value to the electric signals before hand. The signal handling part defines the cumulative sum of the measured electric signals caused by the movements that exceeds the pre set threshold value on a wanted time period. It also evaluates the period that has a risen movement activity grade. The signal handling part also estimates the person's condition and/or physic condition from the following periods average pulse frequency. The control unit divides the measuring time to measurement period's (T) and one or more activity calculator (C(k)) are used in the measurement. The activity calculators are reset to zero in the beginning of the measurement and in case the total value of the signal amplitude exceeds the preset threshold level the activity calculator concerning the period is raised.

12. The device according to the claim 10 having a device attached to it so the person can call for nurse independently.

13. The device according to the claim 12 having a possibility to form a two-way talking connection between the monitored and the monitor.

14. The device according to the claim 10 means to transfer the registered information to be saved on the server.

15. The device according to the claim 10 having signal handling devices in the signal handling part also for the handling of the information obtained from the oxygen oxide metering unit and/or for transfer to the server.

16. The device according to the claim 10 having several entrances for the sensor elements and a multiplexing between them and an automatic locking system for the channel that gives the strongest signal on the wanted frequency area.
